(19) United States
(12) Patent Application Publication
(10) Pub. No.: US 2007/0133736 A1
(43) Pub. Date: Jun. 14, 2007

(54) Devices, systems, and methods for imaging

(75) Inventors: Yunqiang Chen, Plainsboro, NJ (US); Hao Wu, Greenbelt, MD (US); Tong Fang, Morganville, NJ (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS CORPORATE RESEARCH INC, PRINCETON, NJ (US)

(21) Appl. No.: 11/548,863

(22) Filed: Oct. 12, 2006

Related U.S. Application Data
(60) Provisional application No. 60/727,576, filed on Oct. 17, 2005.

(51) Int. Cl. H05G 1/60; A61B 6/00; G01N 23/00; G21K 1/12

(52) U.S. Cl. 378/5

(57) ABSTRACT

Certain exemplary embodiments comprise a method, which can comprise determining an image of a predetermined physiological structure of a patient. The image can be determined based upon a first set of image data of the predetermined physiological structure of the patient. The image can be based upon a second set of image data of the predetermined physiological structure of the patient. The image can be determined based upon an iteratively adjusted movement of the patient.

### CROSS-REFERENCES TO RELATED APPLICATIONS

This application claims priority to, and incorporates by reference herein in its entirety, pending U.S. Provisional Patent Application Ser. No. 60/727,576 (Attorney Docket No. 2005P18878US), filed 17 Oct. 2005.

### BRIEF DESCRIPTION OF THE DRAWINGS

A wide variety of potential practical and useful embodiments will be more readily understood through the following detailed description of certain exemplary embodiments, with reference to the accompanying exemplary drawings in which:

FIG. 1 is a block diagram of an exemplary embodiment of a system 1000;

FIG. 2 is a flowchart of an exemplary embodiment of a method 2000;

FIG. 3 is an exemplary embodiment of an image formation model;

FIG. 4 is a diagram of exemplary Gaussian pyramid strictures;

FIG. 5 is a set of exemplary images of a dual energy chest x-ray;

FIG. 6 is an exemplary set of resulting images; and

FIG. 7 is an exemplary set of resulting images.

### DETAILED DESCRIPTION

Certain exemplary embodiments comprise a method, which can comprise determining an image of a predetermined physiological structure of a patient. The image can be determined based upon a first set of image data of the predetermined physiological structure of the patient. The image can be based upon a second set of image data of the predetermined physiological structure of the patient. The image can be determined based upon an iteratively adjusted movement of the patient.

Certain exemplary embodiments comprise a method, which can comprise determining an image of a bone-only or soft-tissue-only image of a predetermined physiological structure of a patient. The image can be determined based upon two sets of input images acquired with X-Ray of different spectra on the predetermined physiological structure of the patient. The image can be determined based upon an iterative method to compensate the movement of the predetermined physiological structure of a patient during the acquisition of these two sets of images.

In certain exemplary embodiments, two images might be desired of a particular structure of a predetermined portion of an object, such as a physiological structure of a medical patient. In embodiments regarding the physiological structure of the medical patient, a bone only image that is substantially devoid of rendered soft tissue might be desired. Instead of and/or in addition to the bone only image, a soft tissue image might be desired that is substantially devoid of rendered bone. In certain exemplary embodiments, a relatively high energy spectrum can be used as an imaging technique to determine a first image, such as a substantially bone image. In certain exemplary embodiments, a relatively low energy spectrum can be used as an imaging technique to determine a second image, such as a substantially soft tissue image. The first image can comprise artifacts and/or a rather faint and/or blurry version of the second image, and/or the second image can comprise artifacts and/or a rather faint and/or blurry version of the first image. For example, the bone image can comprise soft tissue artifacts and/or the soft tissue image can comprise bone artifacts.

Certain exemplary embodiments can be adapted to utilize data associated with the first image to filter and/or subtract first image artifacts from the second image and/or the second data associated with the second image to filter and/or subtract second image artifacts from the first image. In certain exemplary embodiments, the object, such as the patient, can be at a different location in the second image as compared to the first image. Such a movement can result in motion artifacts as data associated with the first image is used to filter and/or subtract first image artifacts from the second image and/or data associated with the second image is used to filter and/or subtract second image artifacts from the first image. Certain exemplary embodiments can be adapted to iteratively determine a best estimate of the movement of the object based upon an initial iterative estimate of the first image and/or the second image. Certain exemplary embodiments can be adapted to utilize the best estimate of the movement of the object in an iterative determination of a best estimate of the first image and/or a best estimate of the second image.

The disclosure presents exemplary embodiments regarding X-ray imaging of patients. Additional embodiments can be realized in CT imaging, PET imaging, SPECT imaging, magnetic resonance imaging, radar imaging, laser imaging, sonar imaging, and/or any other imaging technology of animate and/or inanimate objects wherein images differ based upon energy and/or frequency spectra and image filtering and/or subtraction is desired, such as when physical movement of the object has occurred between the time the first image is generated and the time the second image is generated.

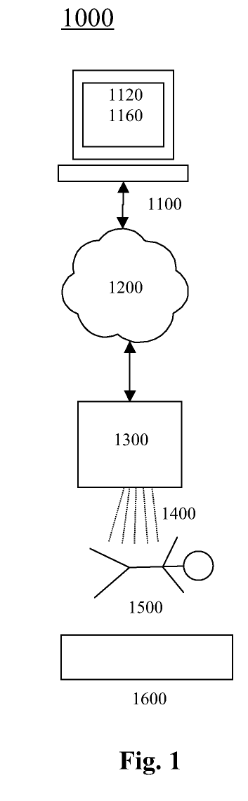

FIG. 1 is a block diagram of an exemplary embodiment of a system 1000, which can comprise an imaging device 1300. Imaging device 1300 can be any device adapted to provide an image, such as an image of a patient 1500. For example, imaging device 1300 can be an X-ray imaging device, and/or a computed tomography (CT) device. Imaging data can be obtained regarding patient 1500, such as via imaging device 1300, a device communicatively coupled thereto, and/or an independent detector 1600, utilizing reflected and/or absorbed emissions 1400 from imaging device 1300.

Imaging device 1300 and/or independent detector 1600 can be communicatively coupled to an information device 100 directly and/or via a network 1200. Information device 1100 can comprise a user program 1160, which can be adapted to analyze, process, manage, align, and/or enhance image data from imaging device 1300. Information device 1100 can comprise a user interface 1120, which can be adapted to render image information associated with imaging device 1300.

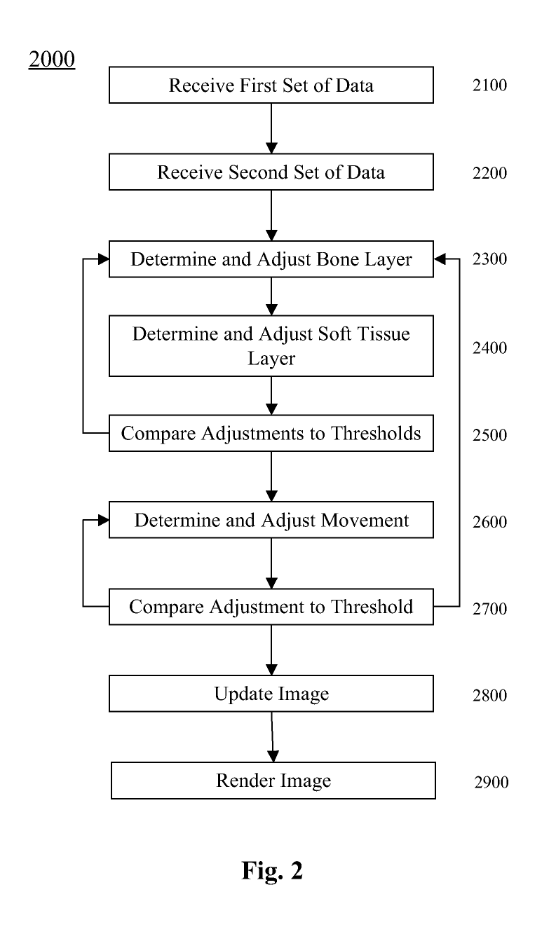

FIG. 2 is a flowchart of an exemplary embodiment of a method 2000. At activity 2100, a first set of image data can be obtained and/or received from an imaging device, such as an X-ray device and/or a detector and/or an information device communicatively coupled thereto. The first set of image data can be of a predetermined physiological structure of a patient. For example, the physiological structure can be a head, neck, foot, leg, thigh, pelvic region, hip region, torso, abdominal region, neck, and/or spinal column, etc. of the patient. The patient can be any animal, such as a human, horse, cow, dog, cat, dolphin, fish, monkey, antelope, and/or bear, etc. The first set of image data can originate from the X-ray device. The X-ray device can be operated at a first energy spectrum. The first set of image data can have originated during a first time interval.

At activity 2200, a second set of image data can be obtained and/or received from the imaging device. The second set of data can be of the predetermined physiological structure and can be received from the imaging device and/or the detector and/or the information device communicatively coupled thereto. The second set of image data can be of the predetermined physiological structure of the patient. The second set of image data can originate from the X-ray device. The X-ray device operated at a second energy spectrum. The second set of image data can have originated during a second time interval. The second time interval can be distinct from the first time interval.

At activity 2300, a mathematical representation of a bone layer can be determined and/or adjusted. The mathematical representation of the bone layer can be determined, such as from prior knowledge about the statistical properties of the bone layer and/or based upon an identification of the physiological structure of the patient. The mathematical representation of the bone layer can be iteratively adjusted, based upon a movement of the patient in the second time interval relative to the first time interval, until the adjustment of the mathematical representation of the bone layer is below a first predetermined threshold. The mathematical representation of the bone layer can be adjusted and/or determined repeatedly, with each repetition based upon an iteration of an adjustment of a mathematical representation of a soft tissue layer. The adjustment of the mathematical representation of the bone layer can be repeated for each of a plurality of iteratively determined estimates of the mathematical representation of the soft tissue layer.

In certain exemplary embodiments, the mathematical representation of the bone layer satisfies some constraints which can be enforced by attempting to minimize one or more cost functions, such as following expression:
−log P(B)∝((1−e)∥B− B̅∥²+λₑe)
where:
P(B) is a probability that the mathematical representation of the bone layer is correct;
e is a binary indicator of whether a pixel is located on an edge of the image;
B is the mathematical representation of the bone layer;
B̅ is an average characteristic of bone within a predetermined neighborhood; and
λₑ is a predetermined factor adapted to penalize edge points.

At activity 2400, the mathematical representation of the soft tissue layer can be determined and/or adjusted. The mathematical representation of the soft tissue layer can be determined, such as from prior knowledge about the statistical properties of the soft tissue layer and/or based upon an identification of the physiological structure of the patient. The mathematical representation of the soft tissue layer can be iteratively adjusted, based upon the movement of the patient in the second time interval relative to the first time interval, until the adjustment of the mathematical representation of the soft tissue layer is below a second predetermined threshold. The mathematical representation of the soft tissue layer can be determined repeatedly, with each repetition based upon an iteration of the adjustment of the mathematical representation of the bone layer. The adjustment of the mathematical representation of the soft tissue layer can be repeated for each of a plurality of iteratively determined estimates of the mathematical representation of the bone layer.

In certain exemplary embodiments the mathematical representation of the soft tissue layer and/or the mathematical representation of the hone layer can be determined based upon constraints and/or joint moments shared between the mathematical representation of the bone layer and the mathematical representation of the soft tissue layer

In certain exemplary embodiments, the mathematical representation of the soft tissue layer satisfies some constraints which can be enforced by attempting to minimize one or more cost functions, such as following expression:
−log P(S)∝((1−e′)∥S− S̅∥²+λ′ₑe′)
where:
P(S) is a probability that the mathematical representation of the soft tissue layer is correct;
S is the mathematical representation of the soft tissue layer;
e′ is a binary indicator of whether a pixel is located on an edge of the soft tissue layer;
S̅ is an average characteristic of soft tissue within a predetermined neighborhood; and
λ′ₑ is a predetermined factor adapted to penalize edge points.

In certain exemplary embodiments, the mathematical representation of the bone layer and the mathematical representation of the soft tissue layer can be determined by attempting to minimize a cost functional:

$$C = {‖ {I}_{1} - a \cdot B - b \cdot S ‖}^{2} + {‖ {I}_{2} - c \cdot T ( B ) - d \cdot T ( S ) ‖}^{2} + {λ}_{1} ( ( 1 - e ) {‖ B - \overline{B} ‖}^{2} + {λ}_{e} e + ( 1 - {e}^{′} ) {‖ S - \overline{S} ‖}^{2} + {λ}^{′} , {e}^{′} ) + {λ}_{3} M I ( B , S )$$

where:
C is a cost associated with the mathematical representation of the bone layer and the mathematical representation of the soft tissue layer and the movement of the patient;
∥ ∥ denotes a norm of a vector;
I₁ is an image based upon the first set of image data;
I₂ is an image based upon the second set of image data;
a is a first constant reflecting attenuation of hone and/or soft tissue to X-rays over a predetermined spectrum;
B is the mathematical representation of the bone layer;
b is a second constant reflecting attenuation of bone and/or soft tissue to X-rays over the predetermined spectrum;
S is the mathematical representation of the soft tissue layer;
c is a third constant reflecting attenuation of bone and/or soft tissue to X rays over the predetermined spectrum;
T(B) is a measure of the adjusted movement of the patient related to the mathematical representation of the bone layer;
d is a fourth constant reflecting attenuation of bone and/or soft tissue to X rays over the predetermined spectrum;
T(S) is a measure of the adjusted movement of the patient related to the mathematical representation of the soft tissue layer;
λ₁ is a first predetermined constraint weighting factor;
e is a binary indicator of whether a pixel is located on an edge of the bone layer;
B̅ is an average characteristic of bone within a predetermined neighborhood;
λₑ is a predetermined factor adapted to penalize edge points in bone layer;
e′ is a binary indicator of whether a pixel is located on an edge of the soft tissue layer;
S̅ is an average characteristic of soft tissue within the predetermined neighborhood;
λ′ₑ is a predetermined factor adapted to penalize edge points in soft tissue layer;
λ₃ is a third predetermined constraint weighting factor; and
MI(B,S) is a function adapted to indicate mutual information and/or other type of correlations such as joint moments shared between the mathematical representation of the bone layer and the mathematical representation of the soft tissue layer.

At activity 2500, an adjustment value associated with the bone layer can be compared to a first predetermined threshold. An adjustment value associated with the soft tissue layer can be compared to a second predetermined threshold. In certain exemplary embodiments the adjustment and/or threshold comparison of the bone layer and the adjustment and/or threshold comparison of the soft tissue layer can take place in separate algorithms and/or a common algorithm.

At activity 2600, a movement of the patient can be determined, adjusted, and/or estimated. An estimate of the movement of the patient can be adjusted until the adjustment of the movement of the patient is below a third predetermined threshold. The estimated movement of the patient can be based upon the adjusted mathematical representation of the bone layer and/or the adjusted mathematical representation of the soft tissue layer. In certain exemplary embodiments, the adjusted movement of the patient can be determined via an updated movement of a Gaussian pyramid, a determination of a control mesh that attempts to minimize a cost, and/or a bilinear interpolation of control points of the mathematical representation of the bone layer and the mathematical representation of the soft tissue layer.

In certain exemplary embodiments, the adjusted movement of the patient is determined via an attempted minimization of an equation:
∥I₂−c·T(B)−d·T(S)∥²+λ₂∥T− T̅∥²
where:
I₂ is an image based upon the second set of image data;
c is a first constant reflecting attenuation of bone and/or soft tissue to X-rays over a predetermined spectrum;
T(B) is a measure of the adjusted movement of the patient related to the mathematical representation of the bone layer;
d is a second constant reflecting attenuation of hone and/or soft tissue to X-rays over the predetermined spectrum;
T(S) is a measure of the adjusted movement of the patient related to the mathematical representation of the soft tissue layer;
λ₂ is a predetermined constraint weighting factor;
T is the adjusted movement of the patient; and
T̅ is an average adjusted movement of the patient.

At activity 2700, an adjustment value of the movement of the patient can be compared to a third predetermined threshold. In certain exemplary embodiments, the adjusting the adjusted mathematical representation of the bone layer, the mathematical representation of the soft tissue layer, and/or the adjusting the movement of the patient can be repeated until the adjustment of the mathematical representation of the bone layer is below the first predetermined threshold, the adjustment of the mathematical representation of the soft tissue layer is less than the second predetermined threshold, and/or the adjustment of the movement of the patient is below the third predetermined threshold. In certain exemplary embodiments, the determination, adjustment, and/or comparison to the third predetermined threshold of the patient movement can occur before, during, or after the determination, adjustment, and/or comparison of a respective adjustment to a respective predetermined threshold of,
the mathematical representation of the bone layer; and/or
the mathematical representation of the soft tissue layer.

At activity 2800, an image associated with the bone layer, the soft tissue layer, and/or the movement can be adjusted and/or updated. The image can be a renderable image and can be automatically determined. The image can be of the predetermined physiological structure of the patient. The image can be determined based upon the iteratively adjusted movement of the patient in the second time interval relative to the first time interval, the adjustment of the mathematical representation of the bone layer, and/or the adjustment of the mathematical representation of a soft tissue layer.

The image can be determined based upon the determined mathematical representation of the bone layer and the determined mathematical representation of the soft tissue layer, wherein each of the determined mathematical representation of the bone layer and the determined mathematical representation of the soft tissue layer can be determined based upon adjusting a cost functional that comprises a mutual information term that comprises bone information and soft tissue information. The image can be determined based upon an iterative algorithm adapted to determine the movement of the patient based upon the determined mathematical representation of the bone layer and the determined mathematical representation of the soft tissue layer.

The image can be determined based upon the determined mathematical representation of the bone layer and the determined mathematical representation of a soft tissue layer and an iterative adjustment of a movement of the patient until the adjustment associated with the movement of the patient is below a predetermined threshold. The cost function can be based upon the mathematical representation of the bone layer and the determined mathematical representation of the soft tissue layer.

At activity 2900, the adjusted and/or updated image of the predetermined physiological structure can be rendered, such as via a user interface. The adjusted and/or updated image can be based upon the adjusted mathematical representation of the bone layer, the adjusted mathematical representation of the soft tissue layer, and/or the adjusted movement of the patient.

Image registration finds various applications in computer vision and medical imaging. Different registration methods can be utilized for rigid or non-rigid movements. Certain exemplary embodiments can be adapted to fuse images from different modalities. Certain exemplary embodiments can be adapted to achieve a relatively robust and accurate registration. Image registration for X-ray dual energy imaging can be challenging due to the overlaid transparent layers (i.e., the bone and soft tissue) and the different appearances between the dual images acquired with X-rays at different energy spectra. Moreover, subpixel accuracy can be desirable for good reconstruction of the bone and soft tissue layers. Certain exemplary embodiments can utilize a coupled Bayesian framework, in which the registration and reconstruction can effectively reinforce each other. With the reconstruction results, accurate matching criteria can be determined for aligning the dual images, instead of treating them as a multi-modality registration. In certain exemplary embodiments, prior knowledge of the bone and soft tissue can be utilized to detect poor reconstruction due to inaccurate registration; and hence correct registration errors in the coupled framework. A multiscale free-form registration algorithm can be implemented to achieve subpixel registration accuracy.

In X-ray dual energy imaging, dual images obtained with X-rays at different energy spectra can have different appearances, which can complicate the designing of appropriate similarity measurements for image alignment. Mutual information can be utilized for multi-modality image registration. Achieving subpixel-accuracy for reconstruction of the bone and soft tissue layers can be a goal of dual energy imaging. Certain exemplary embodiments can utilize a coupled Bayesian method based on the image formation model of dual energy imaging to solve image registration and reconstruction jointly.

Dual energy imaging can improve upon single energy chest radiography, which can have relatively low sensitivity for detecting lung nodules or other subtle details due to the overlap of bone structures and soft tissue. Dual energy imaging can separate the bone and soft tissue from two X-ray images acquired at different energy spectra. Since attenuation coefficients of hone and soft tissue can follow different functions of the energy, the dual images can be weighted and subtracted to acquire separated soft tissue specific and bone specific images, thereby potentially improving evaluations of the lung nodules or pleural calcification.

Image registration can be important in a dual-exposure method, which can perform an image acquisition procedure at two different energy levels in two exposures and provides better image quality than a comparable one-shot method. A time gap between the two exposures can be about 200-300 ms, during which patient or anatomical motions and/or movements might result in motion artifacts in a weighted image subtraction. Certain exemplary embodiments can align the two images before subtraction. The dual images can be overlays of two layers (i.e., bone B and soft tissue S). A potential image formation model can be:
I₁=a·B+b·S
I₂=c·T(B)+d·T(S)
where a, b, c, and d are determinable constants reflecting attenuation coefficients of the bone and soft tissue to the X-ray at different spectra. Based on the two observed images I₁ and I₂, a bone B and soft tissue S can be reconstructed and a non-rigid movement T can be determined.

In certain exemplary embodiments, where there is no substantial movement between the dual images, the bone and soft tissue can be obtained through weighted subtraction of I₁ and I₂:
B=(d·I₁−b·I₂)/(a·d−b·c)
S=(a·I₂−c·I₁)/(a·d−b·c)

In certain exemplary embodiments, image registration can be a preprocessing step to align I₁ and I₂, followed by the weighted subtraction in Equation (2). However, problems that might arise in such a scheme can comprise:
The images I₁ and I₂ might have different appearances due to different attenuation coefficients. A simple similarity measurement might be difficult to determine to guide the registration process. Cross-correlation or mutual information might be used, but might assume only very general dependencies between the two images and neglect the image formation model of Equation (1). Such an approach might encounter difficulties in achieving a relatively robust registration with a relatively high accuracy.
Reconstruction of bone B and soft tissue S can be dependent on the accuracy of the non-rigid registration. Equation (2) indicates that any registration error can be amplified by 1/(a·d−b·c), which might be relatively large when the ratios of coefficients (a/c and b/d) are similar. Exemplary experiments show that subpixel accuracy can be desirable for good reconstruction.
The subtraction procedure in Equation (2) can assume aligned dual images. Registration error might not be corrected via Equation (2). Also, such a subtraction might not utilize prior knowledge of the bone and soft tissue (e.g., smoothness constraint and edge modeling), which might be helpful in image restoration.

Certain exemplary embodiments can comprise a coupled Bayesian framework to register the dual images and reconstruct the bone and soft tissue layers jointly. In the coupled framework, two processes can reinforce each other and can achieve more robust and accurate results. First, with explicit modeling of the bone and soft tissue, relatively accurate similarity measurements can be designed for the registration process instead of treating the dual images as from different modalities and/or relying on more difficult multi-modality registration techniques.

Second, prior knowledge and constraints of the bone and soft tissue can be integrated to check the validity of the reconstruction results. Registration error that causes invalid reconstruction (e.g. highly correlated bone and soft tissue layers) can be detected and/or reduced in the coupled framework. To achieve subpixel accuracy in non-rigid registration, certain exemplary embodiments can comprise a hierarchical free-form registration algorithm with successive accuracy adjustment.

Image registration can be an ill-posed problem, in the sense that image registration can be under-determined and many possible solutions might exist. Image registration can be complicated for non-rigid registration. In certain exemplary embodiments, prior knowledge/constraints of the imaging process and possible movement can be utilized for relatively robust and relatively accurate registration methods.

A Bayesian framework can be utilized to incorporate various constraints based on prior knowledge. An objective of image registration might be to find a most probable movement T that attempts to maximize a posterior probability P(T|I₁,I₂). From Bayesian rule:

$$P ( T \vert {I}_{1} , {I}_{2} ) = \frac{P ( {I}_{1} , {I}_{2} \vert T ) P ( T )}{P ( {I}_{1} , {I}_{2} )}$$

where P(I₁,I₂) is a constant term with respect to T. Therefore, a maximum a posterior (MAP) solution to the registration problem can be obtained as follows:

$$T = arg {max}_{T} log P ( {I}_{1} , {I}_{2} \vert T ) + log P ( T )$$

where P(I₁,I₂|T) defines the similarity measurement to determine how well the movement T aligns the two images. For example, sum of squared difference (SSD) can be used as a single modality and mutual information term. P(T) can represent prior knowledge of the movement field T, e.g. smoothness thereof.

Dual energy imaging can be performed in two steps. First, the dual images can be registered by a multimodality registration method. Then, a weighted subtraction in Equation (2) can be used to reconstruct the underlying bone and soft tissue layers.

A goal of dual energy imaging can be to reconstruct the bone and soft tissue layers. The subtraction procedure might assume no error in the registration. The procedure might attempt to reconstruct B and S to literally match every pixel of I₁ and I₂ based on T. A unique solution of B and S might be found, even if T is not well estimated, and/or no scheme is used to correct the registration error when the reconstruction is not good. Combining registration and reconstruction can be desirable to form a closed loop to adjust the registration result if the reconstructed bone and soft tissue layers are poor (e.g., highly dependent).

Without knowing the bone and soft tissue layers, defining a similarity measurement P(I₁,I₂|T) might be difficult, because the images are generated with X-rays at different energy spectra and have different intensity values even without movement. In certain exemplary embodiments, dual images can be treated as if each image is from a different modality and a similarity measurement can be determined based upon mutual information and/or a cross correlation. Those measurements might neglect the image formation model in Equation (1) and might assume only general dependencies in the dual images. Hence, the similarity measurements can be more susceptible to false matching when dealing with spatial variant bone and soft tissue layers.

In certain exemplary embodiments, registration and reconstruction can be addressed jointly. In certain exemplary embodiments, registration and segmentation can be solved jointly by coupled partial differential equations. Segmentation can be represented by a level-set function to separate the image into exclusive parts. In certain exemplary embodiments, the bone and soft tissue layers might be transparent and overlaid together to generate the acquired images. Hence, certain exemplary embodiments can model the underlying bone and soft tissue layers with two extra appearance templates and handle the layer reconstruction and the registration jointly.

Certain exemplary embodiments can be based on an image formation model of the dual imaging process and/or a coupled framework integrating registration and reconstruction together.

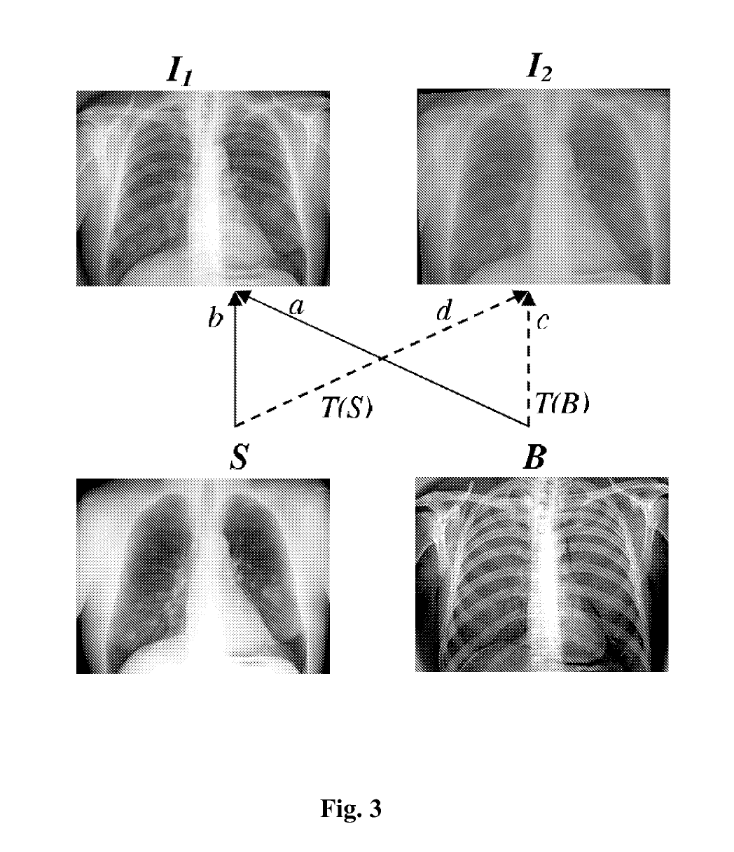

FIG. 3 is an exemplary embodiment of an image formation model, which can comprise dual images generated according to Equation (1). In certain exemplary embodiments, bone B and soft tissue S might not be observed directly. Instead bone B and soft tissue S can be overlaid with different coefficients to form images with different appearances. Considering the hidden variables B and S jointly during the registration process, certain exemplary embodiments can formulate the dual energy image registration as follows:

$$P ( T , B , S \vert {I}_{1} , {I}_{2} ) = \frac{P ( {I}_{1} , {I}_{2} \vert T , B , S ) P ( T , B , S )}{P ( {I}_{1} , {I}_{2} )} \qquad ( 3 )$$

Certain exemplary embodiments can assume that T, B and S are mutually independent, the MAP solution to this problem can be obtained by:

$$T = arg {max}_{T , B , S} log P ( {I}_{1} , {I}_{2} \vert T , B , S ) + log [ P ( T ) P ( B ) P ( S ) ] \qquad ( 4 )$$

This MAP solution leads to a conclusion that the registration and reconstruction can be solved jointly. The movement T, the bone B and the soft tissue S can and/or should be updated together to match the acquired dual images I₁ and I₂.

The prior knowledge about the movement and the appearance of the layers (i.e., P(T), P(B) and P(S)) can be integrated to refine the reconstruction. For example, P(B) and P(S) can model the smoothness constraint with edge modeling and P(T) can regularize the smoothness of the movement field. In certain exemplary embodiments, if the reconstructed B and S do not satisfy the prior knowledge due to registration error, T can be updated and corrected to achieve an acceptable solution.

Furthermore, by introducing bone and soft tissue layers explicitly, the similarity measurement P(I₁,I₂|T,B,S) can be derived. Certain exemplary embodiments might not rely on the mutual information or cross correlation to judge the matching of the dual images even though they have different appearances. Assuming the imaging noise is zero mean Gaussian, a good estimation of B, S and T might allow a synthesis of the dual images and therefore minimize the following cost function:

$$- log P ( {I}_{1} , {I}_{2} \vert T , B , S ) ∝ {‖ {I}_{1} - a \cdot B - b \cdot S ‖}^{2} + {‖ {I}_{2} - c \cdot T ( B ) - d \cdot T ( S ) ‖}^{2}$$

This can be more accurate matching criteria than mutual information or cross correlation and less susceptible to false matching, and hence allows more robust registration with sub-pixel accuracy.

In certain exemplary embodiments, in the proposed coupled Bayesian framework, the registration and reconstruction can reinforce each other and can provide good results.

Based on the proposed coupled Bayesian framework, various prior knowledge or constraints can be utilized and can result in a relatively stable and physically meaningful registration and reconstruction result. In experiments, the following assumptions and constraints were made, which can be true in dual energy imaging:
The movement between the dual images (e.g., patient aspiration) can be modeled by a non-rigid dense movement field T. Certain exemplary embodiments can assume that the movement field is substantially smooth across the image. With this assumption, certain exemplary embodiments can choose the P(T) in Equation (4) as:
   −log P(T)∝∥T− T̅∥²
where T̅ is the average movement within the neighborhood (i.e., the lows pass filtered version of T).
The bone and soft tissue layers can and/or should satisfy the smoothness constraint with edge modeling. Let e be the edge map, where e(p)=1 means the corresponding pixel p is an edge point and the smoothness constraint can be and/or should be suppressed. To prevent all the pixels from being classified as edge points, λₑ can be used to penalize the edge points. Thus:
   −log P(B)∝((1−e)∥B− B̅∥²+λₑe)
   −log P(S)∝((1−e′)∥S− S̅∥²+λ′ₑe′)
where B̅ and S̅ are the average bone and soft tissue within the neighborhood.

Registration error can be a factor in poor reconstruction. In the coupled framework, misalignment can be corrected that can cause reconstruction errors. In certain exemplary embodiments, misaligned dual images cannot cancel out the bone or soft tissue and cause highly correlated artifacts in reconstructed bone and soft tissue layers. Hence, independent analysis between the bone and soft tissue can help detect and correct the registration error. In certain exemplary embodiments, Bayesian frameworks alone might not guarantee the independence constraint even though the independence is used to factorize the joint probabilities. Certain exemplary embodiments can and/or should explicitly enforce the independence by minimizing mutual information MI(B,S).

Using λᵢ to control the weighting of each constraint, an objective function. C, to be minimized can be derived as follows:

$$C = {‖ {I}_{1} - a \cdot B - b \cdot S ‖}^{2} + {‖ {I}_{2} - c \cdot T ( B ) - d \cdot T ( S ) ‖}^{2} + {λ}_{1} ( ( 1 - e ) {‖ B - \overline{B} ‖}^{2} + {λ}_{e} e + ( 1 - {e}^{′} ) {‖ S - \overline{S} ‖}^{2} + {λ}_{e}^{′} {e}^{′} ) + {λ}_{2} {‖ T - \overline{T} ‖}^{2} + {λ}_{3} MI ( B , S ) \qquad ( 5 )$$

The cost function of Equation (5) can be optimized using a variational approach in an iterative manner.

To initialize an exemplary algorithm, a rough registration can be determined based upon Harris corner detection on the dual images. Correspondence can be found based on the cross correlation in the neighborhood around the detected corners. Then the initial movement of each pixel can be approximated by a weighted average of the nearest four corners. Based on this movement field, certain exemplary embodiments can generate an initial reconstruction of the bone and soft tissue, using the weighted subtraction in Equation (2).

Certain exemplary embodiments can fix T and attempt to optimize B and S. For each pixel, certain exemplary embodiments can search within a search range B^{k+1}=B^{k}+δ₁ and S^{k+1}=S^{k+1}δ₂ to find B^{k+1} and S^{k+1} that attempt to minimize the cost function in Equation (5).

Certain exemplary embodiments can then fix B and S and attempt to optimize movement field T. Certain exemplary embodiments can adopt a free-form movement model that is controlled by regularly distributed control points (i.e., rectangular grids). A hierarchical searching strategy can be used to optimize the movement model to achieve sub-pixel accuracy successively as explained in next section in detail. This optimization strategy can be described as following exemplary algorithm:

Algorithm 1: Optimization Scheme

| Data: Given Dual Energy images I₁, I₂. | |
|---|---|
| Result: | Reconstructed Deformation Field T, bone |
| | layer B and soft-tissue layer S. |

Use some traditional geometric based registration
algorithm followed by simple subtraction process to
get T₀, B₀, S₀.
while ˜ stop do
| Fix T^{k}, find the B^{k+1}, S^{k+1}
| for each pixel p do
| | In a search range −m ≦ δ₁ ≦ m,
| | −n ≦ δ₂ ≦ n, for each pixel p, each term in
| | Eq. (5) is computed corresponding to
| | Bₚ^{k} + δ₁, Sₚ^{k} + δ₂ to find the optimal δ₁^{opt}
| | and δ₂^{opt} − Update: Bₚ^{k+1} = Bₚ^{k} + δ₁^{opt};
| | Sₚ^{k+1} = Sₚ^{k} + δ₂^{opt};
| end
| If ||B^{k+1} − B^{k}||² ≦ ε_{B} and
| ||S^{k+1} − S^{k}||² ≦ ε_{S} stop. Otherwise, continue.
| Fix B^{k+1}, S^{k+1}, find T^{k+1} to optimize the cost
| function in Eq. (5) using the hierarchical
| searching strategy described in section 3 and
| Algorithm 2.
| If ||T^{k+1} − T^{k}||² ≦ ε_{T}, stop. Otherwise,
| continue.
end

In the above optimization scheme, the mutual information can be computationally expensive. Certain exemplary embodiments can approximate the entropy term, e.g. H(X)=p(X)log p(X) by Taylor expansion:
x log x=−x₀+(1+log x₀)x+O(x−x₀)²
where the entropy and joint entropy can be approximated by a summation within a neighborhood and hence can be calculated efficiently.

In certain exemplary embodiments, for each iteration, based on the refined bone B^{k} and soft tissue S^{k} from the previous iteration, the registration result T^{k+1} can be further refined.

Certain exemplary embodiments can utilize a non-rigid registration algorithm for Digital Subtraction Angiography based on geometric features. Geometric feature points can be extracted and/or matched based on which triangle meshes are built to define the movement model. However, the geometric features can be dependent on the image content and might not be dense enough and/or accurate enough for dual energy image registration.

Certain exemplary embodiments can utilize a region-based free-form registration algorithm to register breast Magnetic Resonance (MR) images. Regularly distributed control points can be used to control the movement of images for the alignment. The alignment can utilize the information in the image, rather than being based on the sparse feature points. Instead of using cubic B-splines to interpolate the control points, certain exemplary embodiments can use rectangularly spaced control points to control the free-form movement (FFD), which can have better localization and less computation. The performance might be almost the same when the control points are dense enough.

A Gaussian Pyramid structure can be used for the hierarchical searching strategy in certain exemplary embodiments to improve the speed and/or stability of the optimization procedure.

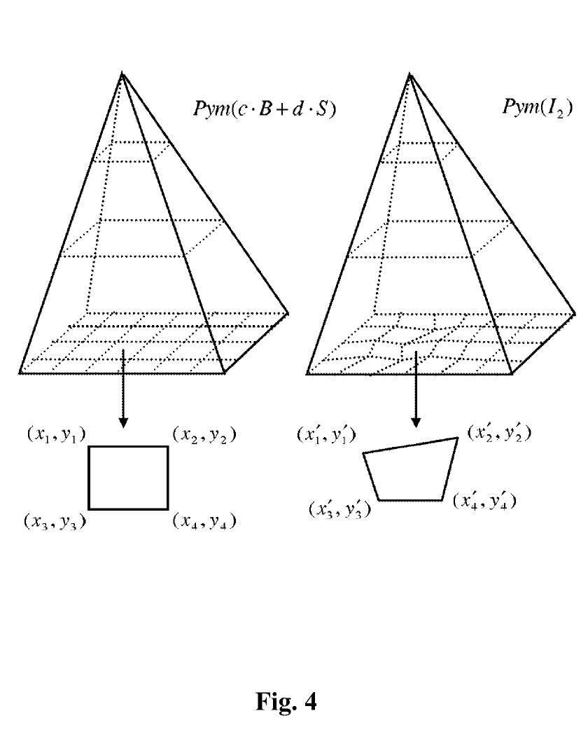

FIG. 4 is a diagram of exemplary Gaussian pyramid structures, which can be utilized in an exemplary movement model and the hierarchical optimization strategy.

The registration can be refined in a coarse-to-fine manner. When a near optimal solution for the coarse level is reached, certain exemplary embodiments can double the density of the control points and map it to the next finer level and further refine the control points based on the finer resolution image. Such a scheme can converge faster and might be less susceptible to local minima.

For each layer, certain exemplary embodiments can deform the bone B and soft tissue S (i.e. c·B+d·S) to match the observed image I₂ by the control points. For each control point, certain exemplary embodiments can search within a local search region to map each control point to a new coordinate in image I₂ and update the matching cost between c—B+d·S and I₂ within the affected regions (each control point affects the four rectangular regions around it). For subpixel accuracy, the search step can be set to be smaller than one pixel. In certain experiments, a value of 0.25 pixels was used as the search step at a finest level.

Suppose the control points on c·B+d·S are at (x₁,y₁), (x₂,y₂), (x₃,y₃), (x₄,y₄) and correspond to (x′₁,y′₁), (x′₂,y′₂), (x′₃,y′₃) and (x′₄,y′₄) in I₂ respectively. The pixel (xₚ,yₚ) inside the grid on c·B+d·S can be mapped to (x′ₚ,y′ₚ) in I₂ based on bilinear interpolation as follows:

$${[ {x}_{p}^{′} , {y}_{p}^{′} ]}^{T} = ( 1 - b ) \cdot {\overline{V}}_{1}^{T} + b \cdot {\overline{V}}_{2}^{T}$$

$$where$$

$$a = ( {y}_{p} - {y}_{1} ) / ( {y}_{2} - {y}_{1} ) , b = ( {x}_{p} - {x}_{1} ) / ( {x}_{3} - {x}_{1} )$$

$${\overline{V}}_{1} = [ 1 - a , a ] [ \begin{matrix}{x}_{1}^{′} & {y}_{1}^{′} \\ {x}_{2}^{′} & {y}_{2}^{′}\end{matrix} ] , {\overline{V}}_{2} = [ 1 - a , a ] [ \begin{matrix}{x}_{3}^{′} & {y}_{3}^{′} \\ {x}_{4}^{′} & {y}_{4}^{′}\end{matrix} ]$$

There are other ways to find the coordinate mapping, such as Homography matrix transformation. In certain experiments, bilinear geometric interpolations were used an appeared to be relatively stable and accurate compared to other transformation methods and potentially more computationally efficient.

Given pixel correspondence, certain exemplary embodiments can compute the cost function ∥I₂−c·T(B)−d·T(S)∥²+λ₂∥T− T̅∥² and find the control mesh that minimizes the cost function. The searching strategy for finding the optimal movement model T can be according to an exemplary Algorithm 2:

Algorithm 2: Hierarchical free-form non-rigid registration

In the Gaussian Pyramid, from coarse level 0 to fine
level N, do the following:
while k ≦ N do
| while ˜ stop do
| | Update deformation Mₖⁱ⁺¹ (x, y) for kth level
| | for each control point Mₖⁱ (x, y) do
| | | Searching in its neighborhood to find the
| | | optimal control mesh that minimizes the
| | | matching cost.
| | | Update Mₖⁱ⁺¹ (x, y) to the optimal
| | | position;
| | end
| | If ||Mₖⁱ⁺¹ − Mₖⁱ||² ≦ εₖ, stop.
| end
| increase the density of control points to generate
| the initial Mₖ₊₁⁰ (x, y), map Mₖⁱ⁺¹ (x, y) to
| Mₖ₊₁⁰ (x, y);
| increase k to k + 1 and reset i to 0;
end

Certain exemplary embodiments can be applied to the X-ray dual energy chest imaging. Experiments and comparisons on both real images and synthesized images showed the improvement of the proposed coupled registration method on registration accuracy and the reconstruction results.

For comparison, certain experiments implemented a separated method, which registered the dual images first and then used weighted subtraction to reconstruct the bone and soft tissue layers. To register the dual images, maximization of mutual information was used to guide the registration. The non-parametric density estimation technique for calculating the joint entropy between the dual images can, to some extent, handle the non-stationary mapping function between the dual images. Certain exemplary embodiments can replace the cost function in Equation (5) by attempting to maximize the mutual information between dual images and apply a similar hierarchical free-form registration method to align the dual images. Based on the registration results, weighted subtraction can be performed to separate the bone and soft tissue layers.

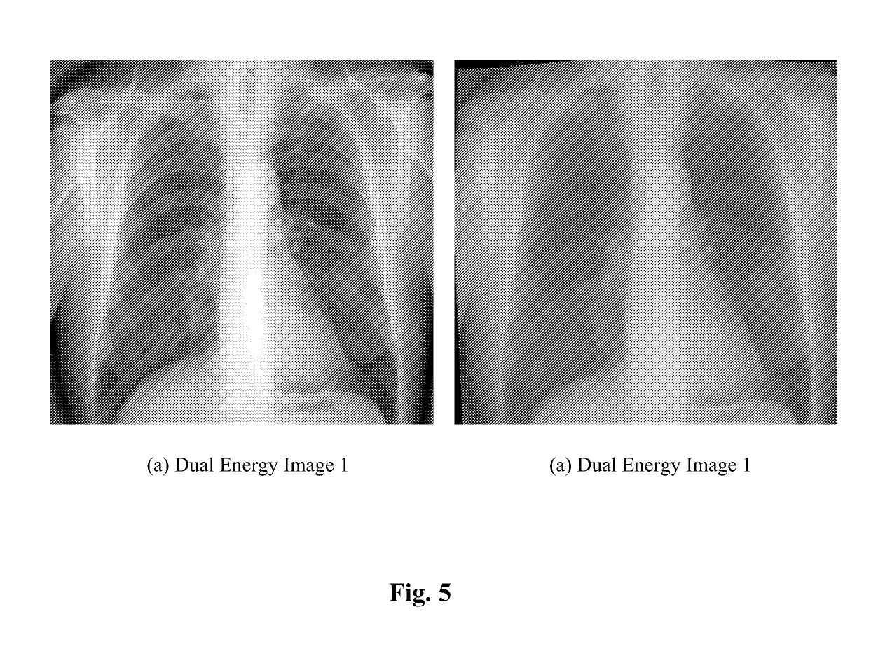

FIG. 5 is a set of exemplary images of a dual energy chest x-ray, which shows the dual images for chest imaging. FIG. 5 illustrates both bone and soft tissue structures overlaid in the images, which can make detecting lung nodules or other subtle details more difficult. Reconstruction of the bone and soft tissue specific images can increase a diagnostic value of the images.

Accurate registration can be desirable for good reconstruction of the bone and soft tissue. Otherwise, the image difference caused by registration error might become much more significant than the different characteristics of the bone and soft tissue.

Certain exemplary experiments compared the separated method with the coupled method. In the separated method, maximization of the mutual information appeared to handle the different appearances between the dual images reasonably well. But when the bone and soft tissue both had complex structures overlaid together, difficulties were experienced in estimating the mapping function robustly and accurately. Also, there was no scheme to refine the registration, even if the reconstruction results did not satisfy prior knowledge in the separated scheme.

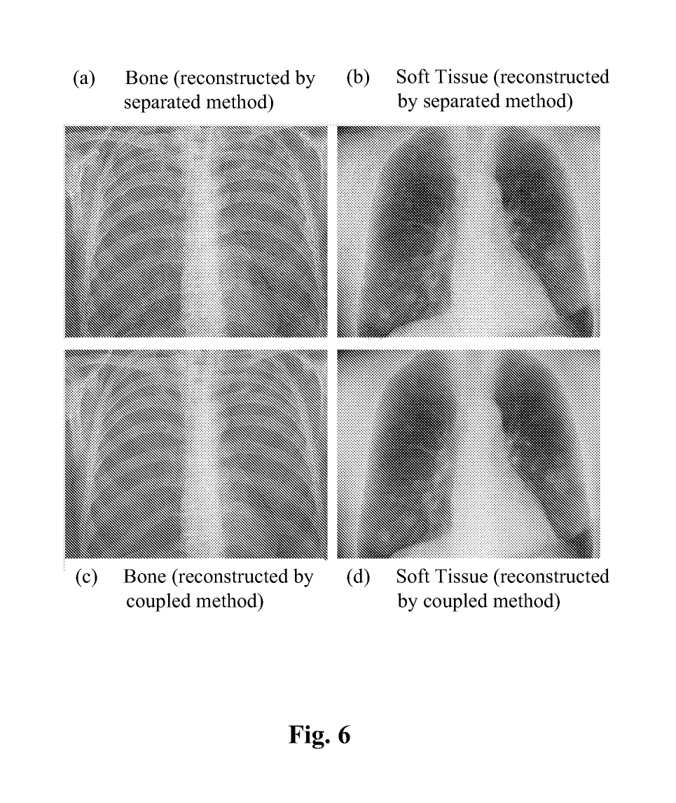

FIG. 6 is an exemplary set of resulting images, which comprises the reconstruction results of the separated method as illustrated in FIG. 6(a) and (b).

FIG. 6 comprises some noticeable artifacts when bone edges and soft tissue structures are overlaid together. The coupled method provides much better results in FIG. 6(c) and (d). The reconstructed results appear to be smoother and cleaner.

To compare the results quantitatively, some tests were performed with synthesized motion to provide ground truth for accurate error analysis. The previous reconstructed bone and soft tissue were selected as ground truth to generate a pair of synthesized images. A transformation field that expands the lung region is applied to simulate an aspiration motion. Quantitative results and comparisons between the separated method and the coupled method are summarized in the following tables.

First the registration accuracy was computed. The estimated movement field T is compared with the ground truth (the synthesized motion). The average and maximum absolute registration error (in pixels) is listed as follows:

| Error in T | Average | Max | Variance |
|---|---|---|---|
| Separated Method | 0.4690 | 1.4783 | 0.0815 |
| Coupled Method | 0.1490 | 1.0182 | 0.0182 |

In the experiments, the separated registration method achieved reasonably good results with maximum registration error of only 1.4783 pixels. However, the proposed coupled framework further improved the registration accuracy and provided consistently better results throughout the image. The mean and the variance of the registration error were smaller in the coupled method.

The error in the reconstructed bone and soft tissue layers were also compared. The absolute difference between the reconstructed results and the ground truth was normalized by the maximal intensity value of the bone and soft tissue images. The errors in different methods are listed in the following table:

| | Average | Max | Variance |
|---|---|---|---|
| Error in B | | | |
| Separated Method | 0.0217 | 0.4766 | 6.05e−4 |
| Coupled Method | 0.0080 | 0.2173 | 1.24e−4 |
| Error in S | | | |
| Separated Method | 0.0145 | 0.3179 | 2.69e−4 |
| Coupled Method | 0.0054 | 0.1451 | 5.49e−5 |

The results indicate that the coupled method generates consistently better reconstruction results.

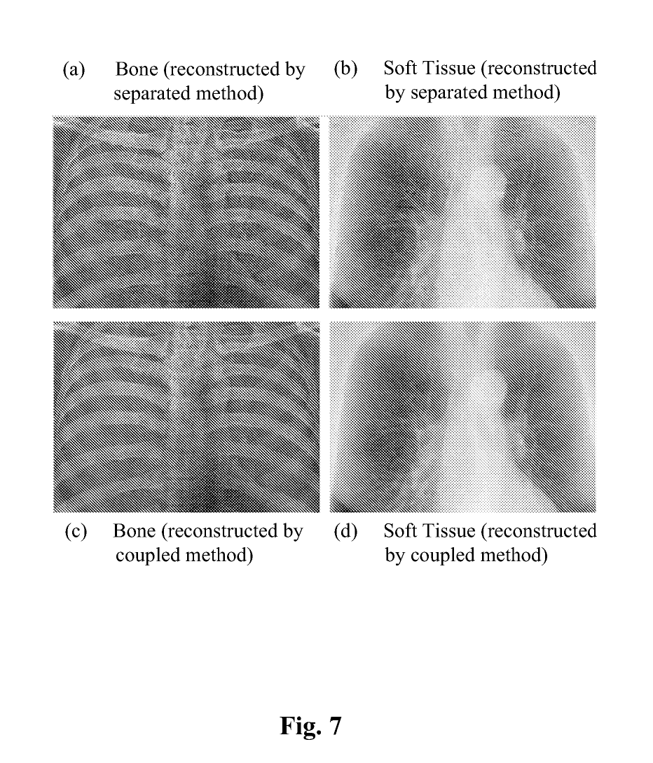

FIG. 7 is an exemplary set of resulting images, which illustrates a comparison on another pair of dual images. The coupled method provided better results on different data set under different imaging conditions.

Certain exemplary embodiments can comprise a coupled Bayesian framework for registering dual energy images and reconstruction of the overlaid bone and soft tissue layers jointly. Certain exemplary embodiments can provide an improvement over the separated scheme where multi-modality image registration is first applied and followed by a simple weighted subtraction to reconstruct the bone and soft tissue. More prior knowledge can be included in an exemplary framework and results in potentially more stable and physically meaningful results.

In certain exemplary embodiments, the coupled algorithm can be helpful for low-dose X-ray imaging to reduce radiation to the patients. In low-dose X-ray imaging, the signal/noise ratio drops significantly.

Definitions

When the following terms are used substantively herein, the accompanying definitions apply. These terms and definitions are presented without prejudice, and, consistent with the application, the right to redefine these terms during the prosecution of this application or any application claiming priority hereto is reserved. For the purpose of interpreting a claim of any patent that claims priority hereto, each definition (or redefined term if an original definition was amended during the prosecution of that patent), functions as a clear and unambiguous disavowal of the subject matter outside of that definition.
a—at least one.
activity—an action, act, deed, function, step, and/or process and/or a portion thereof.
adapted to—suitable, fit, and/or capable of performing a specified function.
adjust—to change so as to match, fit, adapt, conform, and/or be in a more effective state.
adjustment—a magnitude of a change so as to match, fit, adapt, conform, and/or be in a more effective state.
algorithm—a method and/or procedure adapted to solve a problem and/or perform a function.
and/or—either in conjunction with or in alternative to.
apparatus—an appliance or device for a particular purpose
associate—to relate, bring together in a relationship, map, combine, join, and/or connect.
associated with—related to.
attempt—to try to achieve.
attenuation—a decrease in a property.
automatically—acting and/or operating in a manner essentially independent of external human influence and/or control. For example, an automatic light switch can turn on upon “seeing” a person in its view, without the person manually operating the light switch.
average—a value obtained by dividing the sum of a set of quantities by the number of quantities in a set and/or an approximation of a statistical expected value.
based upon—determined in consideration of and/or derived from.
below—less than.
between—in a separating interval and/or intermediate to.
bilinear—interpolation an interpolation of a function of two variables performed via making a linear interpolation first in one direction, and then in the other direction.
binary—characterized by only having a possibility of two outcomes.
bone layer—a representation of bone, the representation substantially devoid of a representation of soft tissue.
can—is capable of, in at least some embodiments.
cause—to bring about, provoke, precipitate, produce, elicit, be the reason for, result in, and/or effect.
characteristic—a distinguishing feature.
comprises—includes, but is not limited to, what follows.
comprising—including but not limited to, what follows.
configure—to design, arrange, set up, shape, and/or make suitable and/or fit for a specific purpose.
constant—continually occurring; persistent; and/or unchanging.
constraint—a limitation.
control mesh—a set of determined data points.
correct—to remedy, adjust in value, and/or change to a more desired value.
cost function—a mathematical expression that establishes an objective measure of closeness of fit between a model and observed data.
create—to make, form, produce, generate, bring into being, and/or cause to exist.
data—information represented in a form suitable for processing by an information device.
data structure—an organization of a collection of data that allows the data to be manipulated effectively and/or a logical relationship among data elements that is designed to support specific data manipulation functions. A data structure can comprise meta data to describe the properties of the data structure. Examples of data structures can include: array, dictionary, graph, hash, heap, linked list, matrix, object, queue, ring, stack, tree, and/or vector.
define—to establish the meaning, relationship, outline, form, and/or structure of; and/or to precisely and/or distinctly describe and/or specify.
movement—an alteration of a form of an entity.
denote—to indicate.
dependent—relying upon and/or contingent upon.
determination—an act of making or arriving at a decision.
determine—to obtain, calculate, decide, deduce, establish, and/or ascertain.
device—an instrumentality adapted to a particular purpose.
distinct—discrete and/or readily distinguishable from all others
during—at some time in a time interval.
each—every one of a group considered individually.
edge—a border at which a surface terminates.
energy—usable power.
equation—a determinable mathematical expression.
estimate—to calculate and/or determine approximately and/or tentatively.
factor—a criteria and/or something that contributes to a cause of an action.
first—before some other thing in an ordering.
fourth—following a third thing in an ordering.
from—used to indicate a source.
functional—a defined mathematical relationship.
further—in addition.
Gaussian pyramid—a hierarchy of low-pass filtered versions of an original image, the versions distributed in a Gaussian manner.
generate—to create, produce, render, give rise to, and/or bring into existence.
haptic—involving the human sense of kinesthetic movement and/or the human sense of touch. Among the many potential haptic experiences are numerous sensations, body-positional differences in sensations, and time-based changes in sensations that are perceived at least partially in non-visual, non-audible, and non-olfactory manners, including the experiences of tactile touch (being touched), active touch, grasping, pressure, friction, traction, slip, stretch, force, torque, impact, puncture, vibration, motion, acceleration, jerk, pulse, orientation, limb position, gravity, texture, gap, recess, viscosity, pain, itch, moisture, temperature, thermal conductivity, and thermal capacity.
image—an at least two-dimensional representation of an entity and/or phenomenon.
indicate—to show, mark, signal, signify, denote, evidence, evince, manifest, declare, enunciate, specify, explain, exhibit, present, reveal, disclose, and/or display.
indicator—one or more signs, tokens, symbols, signals, devices, and/or substance that indicates.
information—facts, terms, concepts, phrases, expressions, commands, numbers, characters, and/or symbols, etc., that are related to a subject. Sometimes used synonymously with data, and sometimes used to describe organized, transformed, and/or processed data. It is generally possible to automate certain activities involving the management, organization, storage, transformation, communication, and/or presentation of information.
information device—any device on which resides a finite state machine capable of implementing at least a portion of a method, structure, and/or or graphical user interface described herein. An information device can comprise well-known communicatively coupled components, such as one or more network interfaces, one or more processors, one or more memories containing instructions, one or more input/output (I/O) devices, and/or one or more user interfaces (e.g., coupled to an I/O device) via which information can be rendered to implement one or more functions described herein. For example, an information device can be any general purpose and/or special purpose computer, such as a personal computer, video game system (e.g., PlayStation, Nintendo Gameboy, X-Box, etc.), workstation, server, minicomputer, mainframe, supercomputer, computer terminal, laptop, wearable computer, and/or Personal Digital Assistant (PDA), iPod, mobile terminal, Bluetooth device, communicator, “smart” phone (such as a Treo-like device), messaging service (e.g., Blackberry) receiver, pager, facsimile, cellular telephone, a traditional telephone, telephonic device, a programmed microprocessor or microcontroller and/or peripheral integrated circuit elements, a digital signal processor, an ASIC or other integrated circuit, a hardware electronic logic circuit such as a discrete element circuit, and/or a programmable logic device such as a PLD, PLA, FPGA, or PAL, or the like, etc.
initialize—to create, produce, render, give rise to, and/or bring into existence.
input/output (I/O) device—an input/output (I/O) device of an information device can be any sensory-oriented input and/or output device, such as an audio, visual, haptic, olfactory, and/or taste-oriented device, including, for example, a monitor, display, projector, overhead display, keyboard, keypad, mouse, trackball, joystick, gamepad, wheel, touchpad, touch panel, pointing device, microphone, speaker, video camera, camera, scanner, printer, haptic device, vibrator, tactile simulator, and/or tactile pad, potentially including a port to which an I/O device can be attached or connected.
interpolation—estimating a value located numerically between two known values.
iteration—a repetition.
iterative—repeatedly.
iteratively—repetitively.
knowledge—the ability to interpret information in order to extract greater meaning,
less than having a measurably smaller magnitude and/or degree as compared to something else.
level—a relative position on a scale and/or a position along a vertical axis indicating height and/or depth.
located—situated in a particular spot and/or position.
location—a place.
machine instructions—directions adapted to cause a machine, such as an information device, to perform one or more particular activities, operations, and/or functions. The directions, which can sometimes form an entity called a “processor”, “kernel”, “operating system”, “program”₅, “application”, “utility”, “subroutine”, “script”, “macro”, “file”, “project”, “module”, “library”, “class”, and/or “object”, etc., can be embodied as machine code, source code, object code, compiled code, assembled code, interpretable code, and/or executable code, etc., in hardware, firmware, and/or software.
machine-readable medium—a physical structure from which a machine, such as an information device, computer, microprocessor, and/or controller, etc., can obtain and/or store data, information, and/or instructions. Examples include memories, punch cards, and/or optically-readable forms, etc.
mathematical representation—an approximation, equivalent, and/or characterization of something based upon a defined action, behavior, procedure, and/or functional relationship.
may—is allowed and/or permitted to, in at least some embodiments.
measure—(n) a quantity ascertained by comparison with a standard. (v) to physically sense, and/or determine a value and/or quantity of something relative to a standard.
medical—of or relating to the study or practice of medicine.
memory device—an apparatus capable of storing analog or digital information, such as instructions and/or data. Examples include a non-volatile memory, volatile memory, Random Access Memory, RAM, Read Only Memory, ROM, flash memory, magnetic media, a hard disk, a floppy disk, a magnetic tape, an optical media, an optical disk, a compact disk, a CD, a digital versatile disk, a DVD, and/or a raid array, etc. The memory device can be coupled to a processor and/or can store instructions adapted to be executed by processor, such as according to an embodiment disclosed herein.
method—a process, procedure, and/or collection of related activities for accomplishing something
minimize—to attempt to reduce in magnitude.
movement—a change in position from one location to another.
mutual—pertaining to each of two or more things.
neighborhood—an area close, adjacent, and/or approximately adjacent to a particular thing.
network—a communicatively coupled plurality of nodes, communication devices, and/or information devices. Via a network, such devices can be linked, such as via various wireline and/or wireless media, such as cables, telephone lines, power lines, optical fibers, radio waves, and/or light beams, etc., to share resources (such as printers and/or memory devices), exchange files, and/or allow electronic communications therebetween. A network can be and/or can utilize any of a wide variety of sub-networks and/or protocols, such as a circuit switched, public-switched, packet switched, connection-less, wireless, virtual, radio, data, telephone, twisted pair, POTS, non-POTS, DSL, cellular, telecommunications, video distribution, cable, terrestrial, microwave, broadcast, satellite, broadband, corporate, global, national, regional, wide area, backbone, packet-switched TCP/IP, IEEE 802.03, Ethernet, Fast Ethernet, Token Ring, local area, wide area, IP, public Internet, intranet, private, ATM, Ultra Wide Band (UWB), Wi-Fi, BlueTooth, Airport, IEEE 802.11, IEEE 802.11a, IEEE 802.11 b, IEEE 802.11g, X-10, electrical power, multi-domain, and/or multi-zone sub-network and/or protocol, one or more Internet service providers, and/or one or more information devices, such as a switch, router, and/or gateway not directly connected to a local area network, etc., and/or any equivalents thereof.
network interface—any physical and/or logical device, system, and/or process capable of coupling an information device to a network. Exemplary network interfaces comprise a telephone, cellular phone, cellular modem, telephone data modem, fax modem, wireless transceiver, Ethernet card, cable modem, digital subscriber line interface, bridge, hub, router, or other similar device, software to manage such a device, and/or software to provide a function of such a device.
norm—a vector function that assigns a positive size to all vectors of a vector space.
operate—to perform a function and/or to work.
optimizing—improving.
originate—to give rise to and/or initiate.
over—with reference to.
packet—a generic term for a bundle of data organized in a specific way for transmission, such as within and/or across a network, such as a digital packet-switching, network, and comprising the data to be transmitted and certain control information, such as a destination address.
patient—a human or other type of animal under supervision for health care purposes.
penalize—to assess a weighting indicative of an estimated cost of an action.
physiological structure—an anatomical part of patient that comprises bone and soft tissue, such as a torso and/or leg; a hierarchy and/or placement of objects in a patient; and/or a manner in which body parts of a patient are organized and/or form a whole.
pixel—a smallest element of an image, and/or a two-dimensional representation thereof, that can be individually processed in a video display system.
plurality—the state of being plural and/or more than one.
point—(n.) a defined physical and/or logical location in at least a two-dimensional system and/or an element in a geometrically described set and/or a measurement or representation of a measurement having a time coordinate and a non-time coordinate.
predetermined—determine, decide, or establish in advance.
prior—preceding in time.
probability—a quantitative representation of a likelihood of an occurrence.
processor—a hardware, firmware, and/or software machine and/or virtual machine comprising a set of machine-readable instructions adaptable to perform a specific task. A processor can utilize mechanical, pneumatic, hydraulic, electrical, magnetic, optical, informational, chemical, and/or biological principles, mechanisms, signals, and/or inputs to perform the task(s). In certain embodiments, a processor can act upon information by manipulating, analyzing, modifying, and/or converting it, transmitting the information for use by an executable procedure and/or an information device, and/or routing the information to an output device. A processor can function as a central processing unit, local controller, remote controller, parallel controller, and/or distributed controller, etc. Unless stated otherwise, the processor can be a general-purpose device, such as a microcontroller and/or a microprocessor, such the Pentium IV series of microprocessor manufactured by the Intel Corporation of Santa Clara, Calif. In certain embodiments, the processor can be dedicated purpose device, such as an Application Specific Integrated Circuit (ASIC) or a Field Programmable Gate Array (FPGA) that has been designed to implement in its hardware and/or firmware at least a part of an embodiment disclosed herein. A processor can reside on and use the capabilities of a controller.
project—to calculate, estimate, or predict.
provide to furnish, supply, give, convey, send, and/or make available.
receive—to gather, take, acquire, obtain, accept, get, and/or have bestowed upon.
recommend—to suggest, praise, commend, and/or endorse.
reflect—to indicate.
related—connected to and/or associated with.
relative—considered with reference to and/or in comparison to something else.
render—to display, annunciate, speak, print, and/or otherwise make perceptible to a human, for example as data, commands, text, graphics, audio, video, animation, and/or hyperlinks, etc., such as via any visual, audio, and/or haptic means, such as via a display, monitor, printer, electric paper, ocular implant, cochlear implant, speaker, etc.
repeat—to do and/or perform again.
repeatedly—again and again; repetitively.
request—(v.) to express a need and/or desire for; to inquire and/or ask for. (n.) that which communicates an expression of desire and/or that which is asked for.
said—when used in a system or device claim, an article indicating a subsequent claim term that has been previously introduced.
second—following a first thing in an ordering.
select—to make and/or indicate a choice and/or selection from among alternatives.
set—a related plurality of predetermined elements; and/or one or more distinct items and/or entities having a specific common property or properties.
share—to use jointly.
sharpness—acuteness.
signal—information, such as machine instructions for activities and/or one or more letters, words, characters, symbols, signal flags, visual displays, and/or special sounds, etc. having prearranged meaning, encoded as automatically detectable variations in a physical variable, such as a pneumatic, hydraulic, acoustic, fluidic, mechanical, electrical, magnetic, optical, chemical, and/or biological variable, such as power, energy, pressure, flowrate, viscosity, density, torque, impact, force, voltage, current, resistance, magnetomotive force, magnetic field intensity, magnetic field flux, magnetic flux density, reluctance, permeability, index of refraction, optical wavelength, polarization, reflectance, transmittance, phase shift, concentration, and/or temperature, etc. Depending on the context, a signal and/or the information encoded therein can be synchronous, asychronous, hard real-time, soft real-time, non-real time, continuously generated, continuously varying, analog, discretely generated, discretely varying, quantized, digital, broadcast, multicast, unicast, transmitted, conveyed, received, continuously measured, discretely measured, processed, encoded, encrypted, multiplexed, modulated, spread, de-spread, demodulated, detected, de-multiplexed, decrypted, and/or decoded, etc.
soft tissue layer—a representation of soft tissue, the representation substantially devoid of a representation of bone.
spectrum—a continuum of entities, as light waves or particles, ordered in accordance with the magnitudes of a common physical property.
step—one of a series of actions, processes, or measures taken to achieve a goal.
store—to place, hold, retain, enter, and/or copy into and/or onto a machine-readable medium.
substantially—to a considerable, large, and/or great, but not necessarily whole and/or entire, extent and/or decree.
system—a collection of mechanisms, devices, data, and/or instructions, the collection designed to perform one or more specific functions.
term—a member comprised by a mathematical representation.
third—following a second thing in an ordering.
threshold—a point that when exceeded produces a given effect or result.
time interval—a quantity and/or finite amount of time between two specified instants, events, and/or states.
transmit—to provide, furnish, supply, send as a signal, and/or to convey (e.g., force, energy, and/or information) from one place and/or thing to another
until—up to a time when.
update—to change.
user interface—a device and/or software program for rendering information to a user and/or requesting information from the user. A user interface can include at least one of textual, graphical, audio, video, animation, and/or haptic elements. A textual element can be provided, for example, by a printer, monitor, display, projector, etc. A graphical element can be provided, for example, via a monitor, display, projector, and/or visual indication device, such as a light, flag, beacon, etc. An audio element can be provided, for example, via a speaker, microphone, and/or other sound generating and/or receiving device. A video element or animation element can be provided, for example, via a monitor, display, projector, and/or other visual device. A haptic element can be provided, for example, via a very low frequency speaker, vibrator, tactile stimulator, tactile pad, simulator, keyboard, keypad, mouse, trackball, joystick, gamepad, wheel, touchpad, touch panel, pointing device, and/or other haptic device, etc. A user interface can include one or more textual elements such as, for example, one or more letters, number, symbols, etc. A user interface can include one or more graphical elements such as, for example, an image, photograph, drawing, icon, window, title bar, panel, sheet, tab, drawer, matrix, table, form, calendar, outline view, frame, dialog box, static text, text box, list, pick list, pop-up list, pull-down list, menu, tool bar, dock, check box, radio button, hyperlink, browser, button, control, palette, preview panel, color wheel, dial, slider, scroll bar, cursor, status bar, stepper, and/or progress indicator, etc. A textual and/or graphical element can he used for selecting, programming, adjusting, changing, specifying, etc. an appearance, background color, background style, border style, border thickness, foreground color, font, font style, font size, alignment, line spacing, indent, maximum data length, validation, query, cursor type, pointer type, autosizing, position, and/or dimension, etc. A user interface can include one or more audio elements such as, for example, a volume control, pitch control, speed control, voice selector, and/or one or more elements for controlling audio play, speed, pause, fast forward, reverse, etc. A user interface can include one or more video elements such as, for example, elements controlling video play, speed, pause, fast forward, reverse, zoom-in, zoom-out, rotate, and/or tilt, etc. A user interface can include one or more animation elements such as, for example, elements controlling animation play, pause, fast forward, reverse, zoom-in, zoom-out, rotate, tilt, color, intensity, speed, frequency, appearance, etc. A user interface can include one or more haptic elements such as, for example, elements utilizing tactile stimulus, force, pressure, vibration, motion, displacement, temperature, etc.

value—a measured, assigned, determined, and/or calculated quantity or quality for a variable and/or parameter.
vector—an expression characterized by a magnitude and a direction.
via—by way of and/or utilizing.
weight—a value indicative of importance.
weighting a measure of importance.
where—in a situation or position.
wherein—in regard to which; and; and/or in addition to.
whether—a conjunction used to introduce the first of two or more alternatives.
within—inside.
x-ray—electromagnetic radiation of non-nuclear origin within the wavelength interval of approximately 0.1 to approximately 100 Angstroms.
   Note

Still other practical and useful embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application.

Thus, regardless of the content of any portion (e.g., title, field, background, summary, abstract, drawing figure, etc.) of this application, unless clearly specified to the contrary, such as via an explicit definition, assertion, or argument, with respect to any claim, whether of this application and/or any claim of any application claiming priority hereto, and whether originally presented or otherwise:
there is no requirement for the inclusion of any particular described or illustrated characteristic, function, activity, or element, any particular sequence of activities, or any particular interrelationship of elements;
any elements can be integrated, segregated, and/or duplicated;
any activity can be repeated, performed by multiple entities, and/or performed in multiple jurisdictions; and
any activity or element can be specifically excluded, the sequence of activities can vary, and/or the interrelationship of elements can vary.

Moreover, when any number or range is described herein, unless clearly stated otherwise, that number or range is approximate. When any range is described herein, unless clearly stated otherwise, that range includes all values therein and all subranges therein. For example, if a range of 1 to 10 is described, that range includes all values therebetween, such as for example, 1.1, 2.5, 3.335, 5, 6.179, 8.9999, etc., and includes all subranges therebetween, such as for example, 1 to 3.65, 2.8 to 8.14, 1.93 to 9, etc.

Any information in any material (e.g., a United States patent, United States patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

Accordingly, the descriptions and drawings are to be regarded as illustrative in nature, and not as restrictive.

What is claimed is:

1. A method comprising:
receiving a first set of image data of a predetermined physiological structure of a patient, said first set of image data originated from an X-ray device operated at a first energy spectrum, said first set of image data originated during a first time interval;
receiving a second set of image data of said predetermined physiological structure of said patient, said second set of image data originated from said X-ray device operated at a second energy spectrum, said second set of image data originated during a second time interval, said second time interval distinct from said first time interval;
determining a mathematical representation of a bone layer of said physiological structure based upon prior knowledge;
determining a mathematical representation of a soft tissue layer of said physiological structure based upon prior knowledge;
based upon a movement of said patient in said second time interval relative to said first time interval, adjusting said mathematical representation of said bone layer and said mathematical representation of said soft tissue layer until said adjustment of said mathematical representation of said bone layer is below a first predetermined threshold and said adjustment of said mathematical representation of said soft tissue layer is less than a second predetermined threshold;
based upon said adjusted mathematical representation of said bone layer and said adjusted mathematical representation of said soft tissue layer, adjusting said movement of said patient until said adjustment of said movement of said patient is below a third predetermined threshold;
repeating said adjusting said adjusted mathematical representation of said bone layer and said mathematical representation of said soft tissue layer and said adjusting said movement of said patient until:
said adjustment of said mathematical representation of said bone layer is below said first predetermined threshold;
said adjustment of said mathematical representation of said soft tissue layer is less than said second predetermined threshold; and
said adjustment of said movement of said patient is below said third predetermined threshold; and
rendering an adjusted image of said predetermined physiological structure of said patient based upon said adjusted mathematical representation of said bone layer, said adjusted mathematical representation of said soft tissue layer, and said adjusted movement of said patient.

2. A method comprising:
automatically determining a renderable image of a predetermined physiological structure of a patient, said image determined based upon a first set of image data of said predetermined physiological structure of said patient, said first set of image data originated from an X-ray device operated at a first energy spectrum, said first set of image data originated during a first time interval, said image based upon a second set of image data of said predetermined physiological structure of said patient, said second set of image data originated from said X-ray device operated at a second energy spectrum, said second set of image data originated during a second time interval, said second time interval distinct from said first time interval, said image determined based upon an iteratively adjusted movement of said patient in said second time interval relative to said first time interval, an adjustment of a mathematical representation of a bone layer, and an adjustment of a mathematical representation of a soft tissue layer until said adjustment associated with optimizing said mathematical representation of said bone layer is below a first predetermined threshold and said adjustment associated with optimizing said mathematical representation of said soft tissue layer is less than a second predetermined threshold, based upon said movement of said patient.

3. The method of claim 2, further comprising:
repeating said adjustment of said mathematical representation of said soft tissue layer for a plurality of iteratively determined estimates of said mathematical representation of said bone layer.

4. The method of claim 2, further comprising:
repeating said adjustment of said mathematical representation of said hone layer for a plurality of iteratively determined estimates of said mathematical representation of said soft tissue layer.

5. The method of claim 2, further comprising:
repeatedly determining said mathematical representation of said bone layer based upon an iteration of said adjustment of said mathematical representation of said soft tissue layer.

6. The method of claim 2, further comprising:
repeatedly determining said mathematical representation of said soft tissue layer based upon an iteration of said adjustment of said mathematical representation of said bone layer.

7. The method of claim 2, further comprising:
determining said mathematical representation of said bone layer based upon prior knowledge about the statistical properties of said bone layer.

8. The method of claim 2, further comprising:
determining said mathematical representation of said soft tissue layer based upon prior knowledge about the statistical properties of said soft tissue layer.

9. The method of claim 2, further comprising:
determining said mathematical representation of said soft tissue layer and determining said mathematical representation of said bone layer based upon joint moments shared between the said mathematical representation of said bone layer and said mathematical representation of said soft tissue layer.

10. The method of claim 2, wherein said mathematical representation of said bone layer and said mathematical representation of said soft tissue layer are determined by attempting to minimize a cost functional:
$$C = {‖ {I}_{1} - a \cdot B - b \cdot S ‖}^{2} + {‖ {I}_{2} - c \cdot T ( B ) - d \cdot T ( S ) ‖}^{2} + {λ}_{1} ( ( 1 - e ) {‖ B - \overline{B} ‖}^{2} + {λ}_{e} e + ( 1 - {e}^{′} ) {‖ S - \overline{S} ‖}^{2} + {λ}_{e}^{′} {e}^{′} ) + {λ}_{3} MI ( B , S )$$
where:
C is a cost associated with said mathematical representation of said bone layer and said mathematical representation of said soft tissue layer and said movement of said patient;
∥ ∥ denotes a norm of a vector;
I₁ is an image based upon said first set of image data;
I₂ is an image based upon said second set of image data;
a is a first constant reflecting attenuation of bone and/or soft tissue to X-rays over a predetermined spectrum;
B is said mathematical representation of said bone layer;
b is a second constant reflecting attenuation of bone and/or soft tissue to X-rays over said predetermined spectrum;
S is said mathematical representation of said soft tissue layer;
c is a third constant reflecting attenuation of bone and/or soft tissue to X-rays over said predetermined spectrum;
T(B) is a measure of said adjusted movement of said patient related to said mathematical representation of said bone layer;
d is a fourth constant reflecting attenuation of bone and/or soft tissue to X-rays over said predetermined spectrum;
T(S) is a measure of said adjusted movement of said patient related to said mathematical representation of said soft tissue layer;
λ₁ is a first predetermined constraint weighting factor;
e is a binary indicator of whether a pixel is located on an edge of said bone layer;
B̅ is an average characteristic of bone within a predetermined neighborhood;
λₑ is a predetermined factor adapted to penalize edge points in bone layer;
e′ is a binary indicator of whether a pixel is located on an edge of said soft tissue layer;
S̅ is an average characteristic of soft tissue within said predetermined neighborhood;
λ′ₑ is a predetermined factor adapted to penalize edge points in soft tissue layer;
λ₂ is a second predetermined constraint weighting factor;
T̅ is an average adjusted movement of said patient;
λ₃ is a third predetermined constraint weighting factor; and
MI(B,S) is a function adapted to indicate mutual information shared between said mathematical representation of said bone layer and said mathematical representation of said soft tissue layer.

11. The method of claim 2, wherein said adjusted movement of said patient is determined via an attempted minimization of an equation:
∥I₂—c·T(B)−d·T(S)∥²+λ₂∥T− T̅∥²
where:
I₂ is an image based upon said second set of image data;
c is a first constant reflecting attenuation of bone and/or soft tissue to X-rays over a predetermined spectrum;
T(B) is a measure of said adjusted movement of said patient related to said mathematical representation of said bone layer;
d is a second constant reflecting attenuation of bone and/or soft tissue to X-rays over said predetermined spectrum;
T(S) is a measure of said adjusted movement of said patient related to said mathematical representation of said soft tissue layer;
λ₂ is a predetermined constraint weighting factor;
T is said adjusted movement of said patient; and
T̅ is an average adjusted movement of said patient.

12. The method of claim 2, wherein said adjusted movement of said patient is determined based upon said mathematical representation of said bone layer and said mathematical representation of said soft tissue layer.

13. The method of claim 2, wherein said adjusted movement of said patient is determined via an updated movement of a Gaussian pyramid.

14. The method of claim 2, wherein said adjusted movement of said patient is determined via a determination of a control mesh that attempts to minimize a cost.

15. The method of claim 2, wherein said adjusted movement of said patient is determined via a bilinear interpolation of control points of said mathematical representation of said bone layer and said mathematical representation of said soft tissue layer.

16. The method of claim 2, wherein said mathematical representation of said bone layer via an attempted minimization of:
−log P(B)∝((1−e)∥B− B̅∥²+λₑe)
where:
P(B) is a probability that said mathematical representation of said bone layer is correct;
e is a binary indicator of whether a pixel is located on an edge of said image;
B is said mathematical representation of said bone layer;
B̅ is an average characteristic of bone within a predetermined neighborhood; and
λₑ is a predetermined factor adapted to penalize edge points.

17. The method of claim 2, wherein said mathematical representation of said soft tissue layer via an attempted minimization of:
−log P(S)∝((1−e′)∥S− S̅∥²+λ′ₑe′).
where:
P(S) is a probability that said mathematical representation of said soft tissue layer is correct;
S is said mathematical representation of said soft tissue layer;
e′ is a binary indicator of whether a pixel is located on an edge of said soft tissue layer;
S̅ is an average characteristic of soft tissue within a predetermined neighborhood; and
λ′ₑ is a predetermined factor adapted to penalize edge points.

18. A method comprising:
automatically determining a renderable image of a predetermined physiological structure of a patient, said image determined based upon a first set of image data of said predetermined physiological structure of said patient, said first set of image data originated from an X-ray device operated at a first energy level, said first set of image data originated during a first time interval, said image based upon a second set of image data of said predetermined physiological structure of said patient, said second set of image data originated from said X-ray device operated at a second energy level, said second set of image data originated during a second time interval, said second time interval distinct from said first time interval, said image determined based upon a determined mathematical representation of a bone layer and a determined mathematical representation of a soft tissue layer and an iterative adjustment of a movement of said patient until said adjustment associated with said movement of said patient is below a predetermined threshold, said cost function based upon said mathematical representation of said bone layer and said determined mathematical representation of said soft tissue layer.

19. A method comprising:
automatically determining a renderable image of a predetermined physiological structure of a patient, said image determined based upon a first set of image data of said predetermined physiological structure of said patient, said first set of image data originated from an X-ray device operated at a first energy level, said first set of image data originated during a first time interval, said image based upon a second set of image data of said predetermined physiological structure of said patient, said second set of image data originated from said X-ray device operated at a second energy level, said second set of image data originated during a second time interval, said second time interval distinct from said first time interval, said image determined based upon a determined mathematical representation of a bone layer and a determined mathematical representation of a soft tissue layer, each of said determined mathematical representation of said bone layer and said determined mathematical representation of said soft tissue layer based upon adjusting a cost functional that comprises a mutual information term that comprises bone information and soft tissue information, said image determined based upon an iterative algorithm adapted to determine a movement of said patient based upon said determined mathematical representation of said bone layer and said determined mathematical representation of said soft tissue layer.

20. A signal comprising machine instructions for activities comprising:
determining a renderable image of a predetermined physiological structure of a patient, said image determined based upon a first set of image data of said predetermined physiological structure of said patient, said first set of image data originated from an X-ray device operated at a first energy spectrum, said first set of image data originated during a first time interval, said image based upon a second set of image data of said predetermined physiological structure of said patient, said second set of image data originated from said X-ray device operated at a second energy spectrum, said second set of image data originated during a second time interval, said second time interval distinct from said first time interval, said image determined based upon an iteratively adjusted movement of said patient in said second time interval relative to said first time interval and an adjustment of a mathematical representation of a bone layer and an adjustment of a mathematical representation of a soft tissue layer until said adjustment associated with optimizing said mathematical representation of said bone layer is below a first predetermined threshold and said adjustment associated with optimizing said mathematical representation of said soft tissue layer is less than a second predetermined threshold, based upon said movement of said patient.

21. A machine-readable medium comprising machine instructions for activities comprising:
determining a renderable image of a predetermined physiological structure of a patient, said image determined based upon a first set of image data of said predetermined physiological structure of said patient, said first set of image data originated from an X-ray device operated at a first energy spectrum, said first set of image data originated during a first time interval, said image based upon a second set of image data of said predetermined physiological structure of said patient, said second set of image data originated from said X-ray device operated at a second energy spectrum, said second set of image data originated during a second time interval, said second time interval distinct from said first time interval, said image determined based upon an iteratively adjusted movement of said patient in said second time interval relative to said first time interval and an adjustment of a mathematical representation of a bone layer and an adjustment of a mathematical representation of a soft tissue layer until said adjustment associated with optimizing said mathematical representation of said bone layer is below a first predetermined threshold and said adjustment associated with optimizing said mathematical representation of said soft tissue layer is less than a second predetermined threshold, based upon said movement of said patient.

22. A system comprising:
a processing means for determining a renderable image of a predetermined physiological structure of a patient, said image determined based upon a first set of image data of said predetermined physiological structure of said patient, said first set of image data originated from an X-ray device operated at a first energy spectrum, said first set of image data originated during a first time interval, said image based upon a second set of image data of said predetermined physiological structure of said patient, said second set of image data originated from said X-ray device operated at a second energy spectrum, said second set of image data originated during a second time interval, said second time interval distinct from said first time interval, said image determined based upon an iteratively adjusted movement of said patient in said second time interval relative to said first time interval and an adjustment of a mathematical representation of a bone layer and an adjustment of a mathematical representation of a soft tissue layer until said adjustment associated with optimizing said mathematical representation of said bone layer is below a first predetermined threshold and said adjustment associated with optimizing said mathematical representation of said soft tissue layer is less than a second predetermined threshold, based upon said movement of said patient; and
a user interface adapted to render said image.
